(19) United States
(12) Patent Application Publication
(10) Pub. No.: US 2008/0175751 A1
(43) Pub. Date: Jul. 24, 2008

(54) Electronic deodorization trash receptacle

(76) Inventors: Yung-Nien Sun, Taichung (TW); Chien-Hsing Lee, Xihu Town (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 11/656,418

(22) Filed: Jan. 23, 2007

(51) Int. Cl. A61L 9/20; A61L 9/00; A61L 9/16

(52) U.S. Cl. 422/5; 422/187; 422/186.3

(57) ABSTRACT

An electronic deodorization trash receptacle includes a shell, a lid, a removable sterilization device and a baffle. The baffle is provided between the shell and lid. The removable sterilization device may be sterilization lamps, with the ultraviolet radiation to provide sterilization and deodorization effects. The baffle can protect users from harm of ultraviolet radiation.

### FIELD OF THE INVENTION

The present invention generally relates to trash receptacles, and more particularly to an electronic deodorization trash receptacle with a sterilization device for sterilizing the interior of the trash receptacle.

### BACKGROUND OF THE INVENTION

Most of conventional trash bins are like general trash bins, but some trash cans have been improved.. For example, TW Appl. No. 87203113 disclosed an ozone trash bin that is equipped with an ozone device on the top and uses high voltage transform circuit and catalytic converter to produce ozone. However the ozone device only has deodorizing and sterilizing effects, if the germs have strong activity, the ozone device is unable to kill the germs. Such device has shortcomings, which are:

1. High cost: the ozone device uses catalytic converter to produce ozone like a car use ozone generator also uses catalytic converter, which needs to be replaced after a period of time, and the price of catalytic converter is high, which costs a lot of money.

2. Limited lifetime: the catalytic converter needs to be replaced after a period of time, which is inconvenient for use.

3. Not easy to replace when damaging: due to the high voltage, it needs professional technique to replace the device when damaging, which is not easy for a general user to replace the device.

### SUMMARY OF THE INVENTION

The primary objective of the present invention is to provide a trash receptacle with a sterilization device having deodorization and sterilization effects. The lid of the trash receptacle is equipped with lamps that can produce both ozone and ultraviolet radiation, by using the ultraviolet radiation irradiating inside the trash receptacle to eliminate the odor and kill bacteria, and the ozone produced thereof to provide a dual sterilization and refresh the air.

The other objective of the present invention is to provide a trash receptacle with a sterilization device having deodorization and sterilization effects. The lid of the trash receptacle has a cavity that is equipped with opposite locking members and propping members for installing a removable sterilization device. The removable sterilization device includes a cover that is equipped with elastic members and barricading members relative to locking members and propping members. The cover has a lamp holder that is equipped with receiving members for installing the sterilization lamp, and two opposite reflectors are provided on top and bottom of the sterilization lamp. With the cover being equipped with elastic member relative to the locking member, the cover can be replaced. Furthermore, while the propping member contacts with the barricading member, which can conduct electricity and activate the sterilizing lamp to produce ultraviolet radiation to irradiate the interior of the trash receptacle. The functions of sterilization and deodorization can be phased, simultaneous or circulating, with which to eliminate the odor of the trash receptacle and kill bacteria. In addition, the objective of convenient replacement is achieved.

The present invention will become more obvious from the following description when taken in connection with the accompanying drawings that show, for purposes of illustration only, a preferred embodiment in accordance with the present invention.

### BRIEF DESCRIPTION OF THE DRAWINGS

FIG. 1 is a perspective view of the present invention;

FIG. 2 is an exploded view of the present invention;

FIG. 3 is a cross-sectional view of the present invention;

FIG. 4 is a cross-sectional view showing the motion of the present invention; and

FIG. 5 is a sterilization and deodorization mode chart of the present invention.

### DETAILED DESCRIPTION OF THE PREFERRED EMBODIMENT

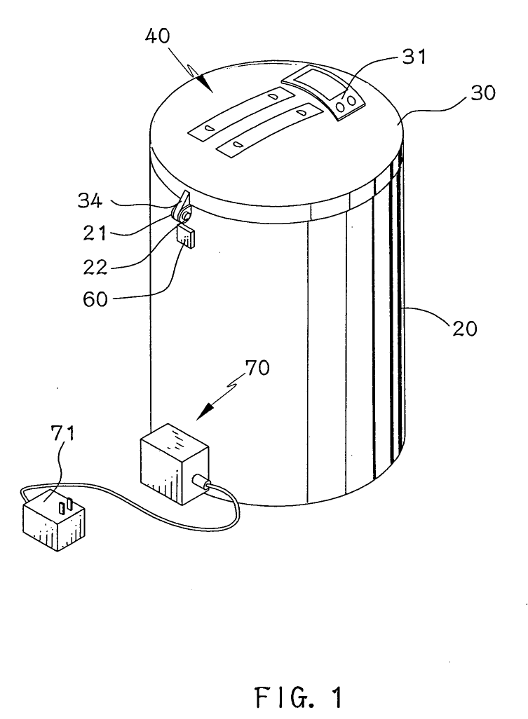

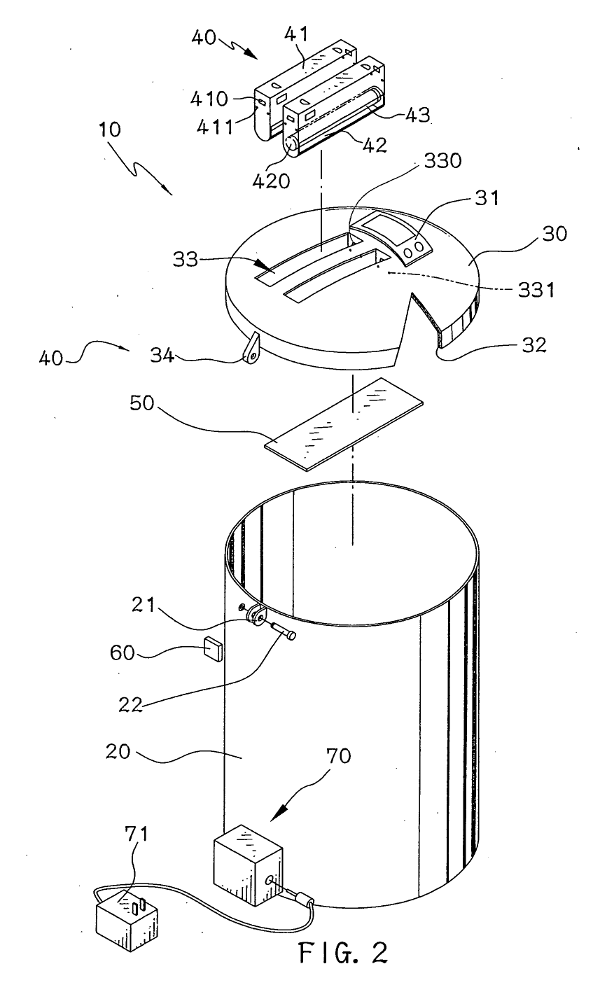

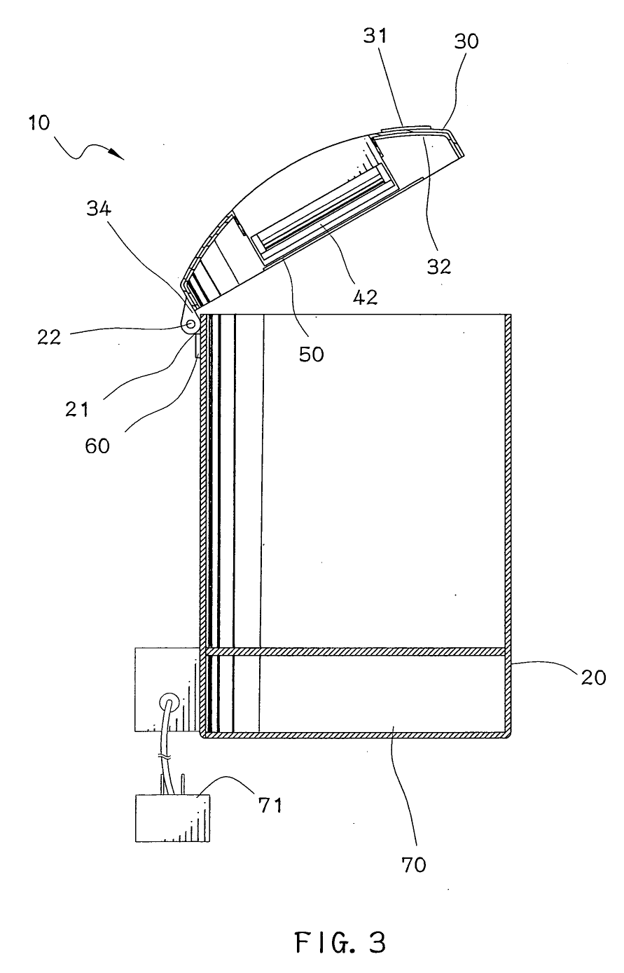

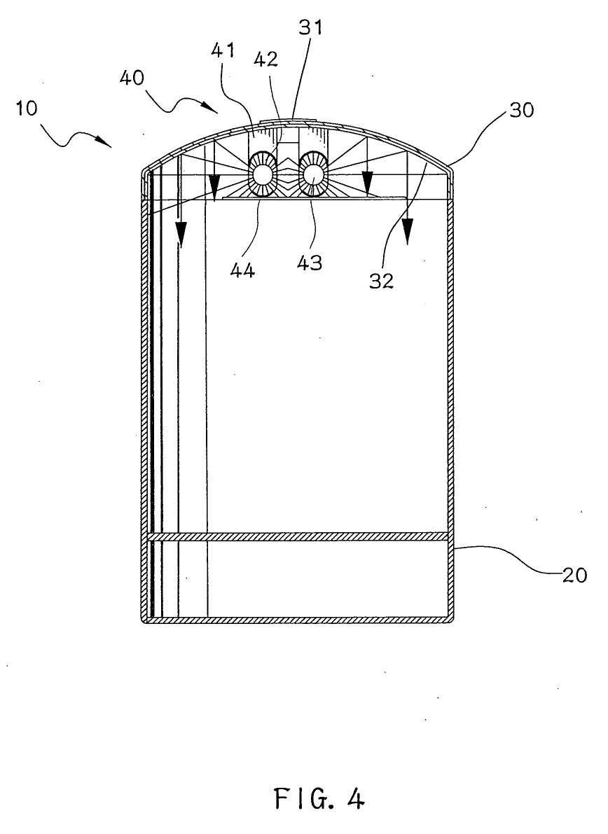

Referring to FIGS. 1 to 4, a trash receptacle 10 is depicted which is constructed in accordance with the principles of the present invention. The trash receptacle 10 has a figure of a conventional trash bin, kitchen waste bin or laundry bin, which includes a shell 20 and a lid 30. An elastic positioning structures which includes a removable sterilization device 40 and a baffle 50 is provided inside the shell 20 and the lid 30, a magnetic switch 60 is provided outside the shell 20, and a power supply 70 for supplying power is provided on the lower portion of the shell 20.

The trash receptacle 10 includes a cylindrical shell 20 that has a round upper pivoting member 21 projected outwardly from the upper portion of the shell 20. The upper pivoting member 21 is connected to the lid 30 with a fixing member 22.

The lid 30 is located on the top of the shell 20, which is a thin annular shell and has the same diameter with the shell 20. A control panel 31 is provided on the top of the lid 30 for controlling a sterilization structure, and a reflective layer 32 is stuck on the inner surface of the lid 30. The lid 30 has at least one long cavity 33 near the control panel 31. The cavity 33 is equipped with elastic positioning structures that are equipped with locking members 330 and propping members 331 for accommodating the removable sterilization device 40. A round lower pivoting member 34 projected outwardly from the periphery of the lid 30 is connected with the upper pivoting member 21 of the shell 20 by a pivot to form a liftable structure.

The removable sterilization device 40 includes at least one cover 41, lamp holder 42 and sterilization lamp 43. The cover 41 is a rectangular shape and the elastic positioning structures on both ends thereof are equipped with elastic members 410 and barricading members 411 which are engaged with the locking members 330 and the propping members 331 of the cavity 33. The cover 41 has a lamp holder 42 that is equipped with receiving members 420 for installing the sterilization lamp 43 that is a tubular lamp. Two reflectors 44 which are in the form of plates are located respectively on top and bottom of the sterilization lamp 43, wherein the reflectors 44 are applied with optical catalyst or catalyst. The sterilization device 40 uses an electronic ballast to stabilize voltage. When the sterilization lamp 43 is activated, the ultraviolet radiation irradiates upon the reflectors 44 and then reflects onto the reflective layer 32, thus the ultraviolet radiation can reflect onto the interior of the shell 20.

The reflective layer 32, reflectors 44 and baffle 50 are applied with optical catalyst or catalyst, with the ultraviolet radiation released from the sterilization lamp 43 having sterilization and deodorization effects, and being able to decompose oxygen into ozone. In addition, the ultraviolet radiation is able to produce a chemical reaction, ionic effect and other chemical reaction with optical catalyst or catalyst, which causes re-decomposition and produces oxygen. Then the objectives of refreshing air, sterilization and deodorization are attained.

A baffle 50 is provided under the removable sterilization device 40, the baffle 50 is a thin opaque plate and able to baffle the ultraviolet radiation emitted directly from the sterilization lamp 43 with its limited area.

The upper hinge 21 is equipped with a magnetic switch 60 that has a timer inside. When the lid 30 of the trash receptacle 10 is opened, the power supply 70 or a plug 71 will be shut off. The power supply 70 can be a rechargeable battery and the plug 71 can be a DC power supply.

A control panel 31 is disposed on the outside of the lid 30, and will display the command of on, off and other setting data for the user to control directly the action of the removable sterilization device 40 inside, the current operational status of the removable sterilization device 40 can also be learned from the display, optimal practicality in provision is attained.

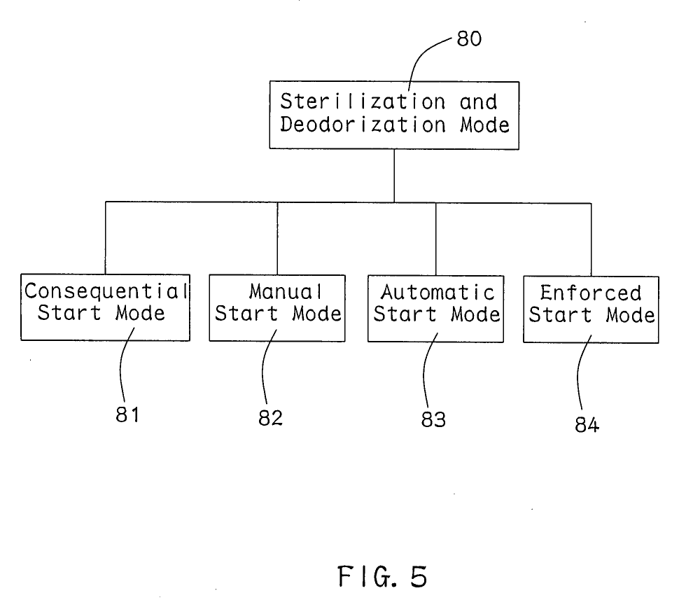

Referring to FIG. 5, which is the sterilization and deodorization mode chart 80 of the trash receptacle 10.

Consequential start mode 81: when the lid 30 of the trash receptacle 10 is closed, by operating the control panel 31 to control one or all the sterilization lamps 43 to produce ultraviolet radiation that irradiates the reflectors 44 automatically and reflects upon the reflective layer 32 of the lid 30 to produce reflection. A part of the reflection irradiates inside the shell 20 to have sterilization and deodorization effects of ultraviolet radiation or ultraviolet radiation cooperating with optical catalyst or catalyst, and other parts of the reflections are stopped by the baffle 50. The sterilization lamp 43 goes off after a period of time. One or all the sterilization lamps 43 are activated again after a period of time and then the ultraviolet radiation or ultraviolet radiation cooperating with optical catalyst or catalyst executes the functions of sterilization and deodorization again, furthermore the functions of sterilization and deodorization can be continuing and circulating.

Manual start mode 82: when the lid 30 of the trash receptacle 10 is opened, the magnetic switch 60 will shut off the power supply 70 or the plug 71 that supplies power. After the lid 30 is closed, by operating the control panel 31 to activate the sterilization lamp 43 to start the functions of sterilization and deodorization immediately, which has no need to wait for the timer of the magnetic switch 60 to start the functions of sterilization and deodorization automatically.

Automatic start mode 83: when the lid 30 of the trash receptacle 10 is opened, the magnetic switch 60 will shut off the power supply 70 or the plug 71 that supplies power. The timer of the magnetic switch 60 will activate the sterilization lamp 43 to start the functions of sterilization and deodorization after the lid 30 of the trash receptacle 10 is closed for a period of time.

Enforced start mode 84: when the lid 30 of the trash receptacle 10 is opened, the magnetic switch 60 will shut off the power supply 70 or the plug 71 that supplies power. After the lid 30 is closed, by operating the control panel 31 to force the sterilization lamp 43 to produce ultraviolet radiation for sterilizing and deodorizing, thus the objective of continuing sterilization and deodorization effects is achieved.

While we have shown and described the embodiment in accordance with the present invention, it should be clear to those skilled in the art that further embodiments may be made without departing from the scope of the present invention.

1-11. (canceled)

12. Sterilization and deodorization modes and methods of an electronic deodorization trash receptacle comprising:
a consequential start mode;
a manual start mode;
an automatic start mode; and
an enforced start mode.

13. The sterilization and deodorization modes and methods of an electronic deodorization trash receptacle as claimed in claim 12, wherein said consequential start mode: when a lid of said trash receptacle being closed, by operating a control panel to control one or all sterilization lamps to produce ultraviolet radiation that irradiates reflectors automatically and reflects upon a reflective layer of said lid to produce reflection, a part of said reflection irradiates inside a shell to have sterilization and deodorization effects of ultraviolet radiation or ultraviolet radiation cooperating with optical catalyst or catalyst, and other parts of said reflections being stopped by a baffle, said sterilization lamp going off after a period of time, one or all said sterilization lamps being activated again after a period of time and then said ultraviolet radiation or ultraviolet radiation cooperating with optical catalyst or catalyst executing functions of sterilization and deodorization again, furthermore said functions of sterilization and deodorization being able to be continuing and circulating.

14. The sterilization and deodorization modes and methods of an electronic deodorization trash receptacle as claimed in claim 12, wherein said manual start mode: when a lid of said trash receptacle being opened, a magnetic switch shutting off a power supply or a plug that supplies power, after said lid being closed, by operating a control panel to activate a sterilization lamp to start functions of sterilization and deodorization immediately, which having no need to wait for a timer of said magnetic switch to start said functions of sterilization and deodorization automatically.

15. The sterilization and deodorization modes and methods of an electronic deodorization trash receptacle as claimed in claim 12, wherein said automatic start mode: when a lid of said trash receptacle being opened, a magnetic switch shutting off a power supply or a plug that supplies power, a timer of said magnetic switch activating said sterilization lamp to start the functions of sterilization and deodorization after said lid of said trash receptacle being closed for a period of time.

16. The sterilization and deodorization modes and methods of an electronic deodorization trash receptacle as claimed in claim 12, wherein said enforces start mode: when a lid of said trash receptacle being opened, a magnetic switch shutting off a power supply or a plug that supplies power, after said lid being closed, by operating a control panel to force a sterilization lamp to produce ultraviolet radiation for sterilizing and deodorizing, thus an objective of continuing sterilization and deodorization effects being achieved.
